(19) United States
(12) Patent Application Publication
(10) Pub. No.: US 2015/0250590 A1
(43) Pub. Date: Sep. 10, 2015

(54) TRANSAPICAL MITRAL CHORDAE REPLACEMENT

(71) Applicant: St. Jude Medical, Cardiology Division, Inc., St. Paul, MN (US)

(72) Inventors: Sara Jane Gries, Osseo, MN (US); Jacob Paul Mertens, Minnetonka, MN (US)

(73) Assignee: ST. JUDE MEDICAL, CARDIOLOGY DIVISION, INC., St. Paul, MN (US)

(21) Appl. No.: 14/632,300

(22) Filed: Feb. 26, 2015

Related U.S. Application Data
(60) Provisional application No. 61/950,292, filed on Mar. 10, 2014.

(51) Int. Cl. A61F 2/24; A61B 17/00

(52) U.S. Cl. CPC A61F 2/2457; A61B 17/00234; A61F 2/2466; A61F 2210/0014; A61B 2017/00243; A61B 2017/00336; A61F 2230/0063; A61F 2220/0008

(57) ABSTRACT

A chordae replacement device includes a first anchor for coupling to a native valve leaflet, a second anchor for coupling to heart tissue and a filament adapted for connection between the first anchor and the second anchor so as to limit the movement of the native valve leaflet away from the second anchor.

### CROSS-REFERENCE TO RELATED APPLICATIONS

The present application claims the benefit of the filing date of U.S. Provisional Patent Application No. 61/950,292 filed Mar. 10, 2014, the disclosure of which is hereby incorporated herein by reference.

### BACKGROUND OF THE INVENTION

The present invention relates to heart valve repair and, in particular, to mitral valve leaflet repair. More particularly, the present invention relates to devices and methods for replacing chordae tendineae.

Properly functioning heart valves can maintain unidirectional blood flow in the circulatory system by opening and closing, depending on the difference in pressure from one side of the valve to the other. The two atrioventricular valves (mitral and tricuspid valves) are multicusped valves that prevent backflow from the ventricles into the atria during systole. They are anchored to the wall of the ventricle by chordae tendineae, which prevent the valve from inverting.

The mitral valve is located at the gate of the left ventricle and is made up of two leaflets and a diaphanous incomplete ring around the valve, known as the mitral valve annulus. When the valve opens, blood flows into the left ventricle. After the left ventricle fills with blood and contracts, the two leaflets of the mitral valve are pushed upwards and close, preventing blood from flowing back into the left atrium and the lungs.

Mitral valve prolapse is a type of myxomatous valve disease in which the abnormal mitral valve leaflets prolapse (i.e., a portion of the affected leaflet may be billowed, loose, and floppy). Furthermore, the chordae tendineae may stretch and thus become too long, or the chordae tendineae may be ruptured. As a result, the valve does not close normally and the unsupported valve leaflet may bulge back, or “prolapse,” into the left atrium like a parachute. Thus, as the ventricle contracts, the abnormal leaflet may be propelled backwards, beyond its normal closure line and into the left atrium, thereby allowing blood to return to the left atrium and the lungs.

Mitral valve prolapse causes mitral regurgitation. Isolated posterior leaflet prolapse of the human heart mitral valve, i.e., prolapse of a single leaflet, is the most common cause of mitral regurgitation. The exact cause of the prolapse is not clear. Untreated mitral regurgitation may lead to congestive heart failure and pulmonary hypertension.

Despite the various improvements that have been made to devices and methods for mitral valve leaflet repair, there remain some shortcomings. For example, conventional methods of treating mitral valve prolapse include replacement of the mitral valve, clipping the two mitral valve leaflets to one another, and resection of the prolapsed segment using open heart surgery. Such surgical methods may be invasive to the patient and may require an extended recovery period.

Therefore, there is a need for further improvements to the current techniques for treating heart valve leaflet prolapse. Among other advantages, the present invention may address one or more of these needs.

### SUMMARY OF THE INVENTION

In some embodiments, a chordae replacement device includes a first anchor for coupling to a native valve leaflet, a second anchor for coupling to heart tissue and a filament adapted for connection between the first anchor and the second anchor so as to limit the movement of the native valve leaflet away from the second anchor.

In some embodiments, a delivery device for implanting a chordae replacement device having a first anchor, a second anchor and a filament at a deployment site in a patient includes an outer shaft, an inner sheath disposed within the outer shaft and translatable relative to the outer shaft, the inner sheath being coupleable to the first anchor and a piercing wire disposed within the inner sheath and translatable relative to the inner sheath.

In some embodiments, a method is described of deploying a chordae replacement device at a target site, the chordae replacement device including a first anchor for coupling to a native valve leaflet, a second anchor for coupling to heart wall tissue and a filament having one end connected to the first anchor, and a free end. A delivery device is introduced to the target site, the delivery device including an outer shaft, an inner sheath disposed within the outer shaft and translatable relative to the outer shaft, and a piercing wire disposed within the inner sheath and translatable relative to the inner sheath. The native valve leaflet may be penetrated with the piercing wire and the first anchor advanced over the piercing wire to deploy the first anchor in engagement with the native valve leaflet. The delivery system may be withdrawn toward the heart wall and the second anchor deployed in engagement with the heart wall. A filament may be connected to the second anchor to define a fixed length of filament between the first anchor and the second anchor.

### BRIEF DESCRIPTION OF THE DRAWINGS

Various embodiments of the present invention are disclosed herein with reference to the drawings, wherein:

FIG. 1 is a schematic representation of a human heart showing a transapical delivery approach;

FIG. 2A is a schematic representation of a native mitral valve and associated structures during normal operation;

FIG. 2B is a schematic representation of a native mitral valve having a prolapsed leaflet;

FIG. 3 is a schematic representation of a chordae replacement device having two anchors and a filament;

FIG. 4 is an enlarged view of the first anchor of the chordae replacement device;

FIG. 5 is an enlarged view of the second anchor of the chordae replacement device;

FIGS. 6A-6G are schematic representations showing the steps of using a delivery device to deploy the chordae replacement device of FIG. 3 within a patient;

FIG. 7 illustrates a chordae replacement device that has been anchored at two ends; and

FIG. 8 illustrates the final placement of a chordae replacement device after anchoring the two ends and tensioning of the filament.

Various embodiments of the present invention will now be described with reference to the appended drawings. It is to be appreciated that these drawings depict only some embodiments of the invention and are therefore not to be considered limiting of its scope.

### DETAILED DESCRIPTION OF THE INVENTION

Blood flows through the mitral valve from the left atrium to the left ventricle. As used herein, the term “inflow,” when used in connection with a mitral heart valve, refers to the end of the heart valve closest to the left atrium when the heart valve is implanted in a patient, whereas the term “outflow,” when used in connection with a mitral heart valve, refers to the end of the heart valve closest to the left ventricle when the heart valve is implanted in a patient. When used in connection with devices for delivering a chordae replacement device into a patient, the terms “trailing” and “leading” are to be taken as relative to the user of the delivery devices. “Trailing” is to be understood as relatively close to the operator, and “leading” is to be understood as relatively farther away from the operator.

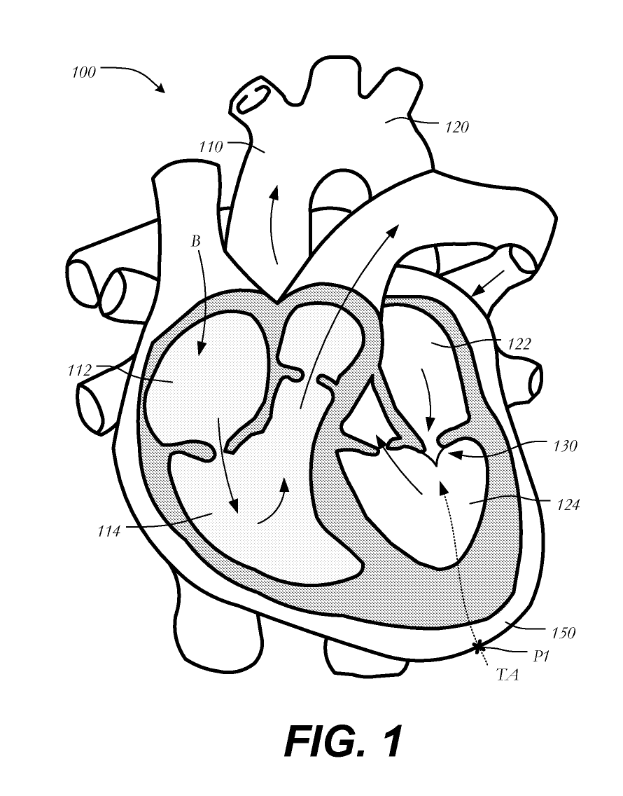

FIG. 1 is a schematic representation of a human heart 100. The human heart includes two atria and two ventricles: a right atrium 112 and a left atrium 122, and a right ventricle 114 and a left ventricle 124. As illustrated in FIG. 1, the heart 100 further includes an aorta 110, and an aortic arch 120. Disposed between the left atrium and the left ventricle is the mitral valve 130. The mitral valve 130, also known as the bicuspid valve or left atrioventricular valve, is a dual-flap that opens as a result of increased pressure from the left atrium as it fills with blood. As atrial pressure increases above that of the left ventricle, the mitral valve opens and blood passes toward the left ventricle. Blood flows through heart 100 in the direction shown by arrows “B”.

A dashed arrow, labeled as “TA”, indicates a transapical approach for repairing or replacing heart valves such as a mitral valve. In transapical delivery, a small incision is made between the ribs and into the apex of the left ventricle 124 at position “P1” in heart wall 150 to deliver a prosthesis or device to the target site.

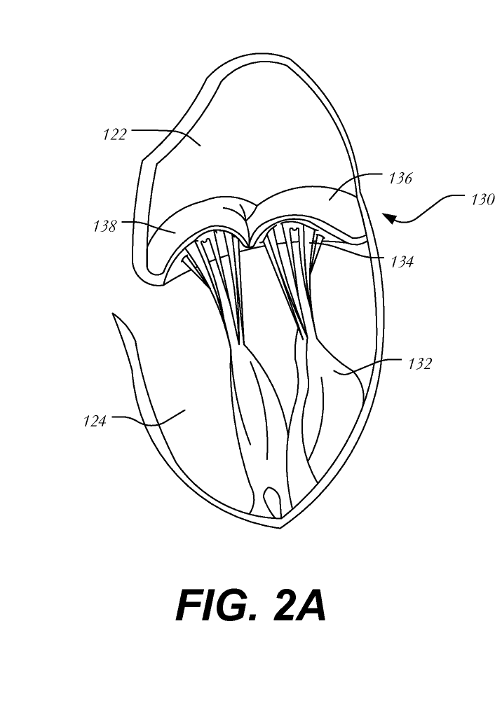

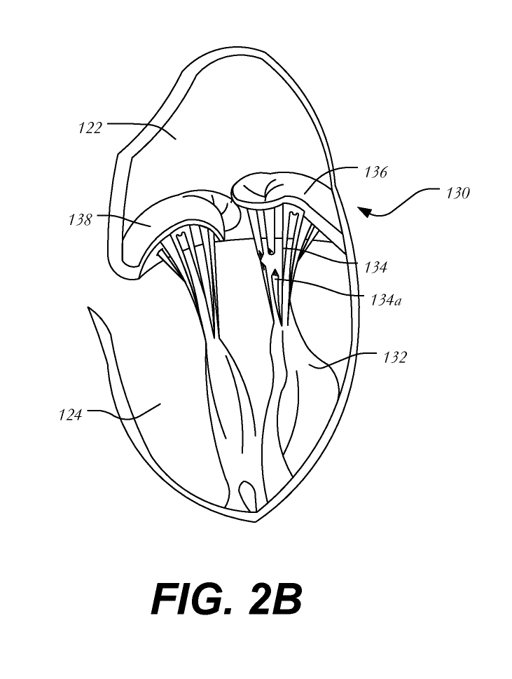

FIG. 2A is a more detailed schematic representation of a native mitral valve 130 and its associated structures. As previously noted, mitral valve 130 includes two flaps or leaflets, a posterior leaflet 136 and an anterior leaflet 138, disposed between left atrium 122 and left ventricle 124. Cord-like tendons known as chordae tendineae 134 connect the two leaflets 136, 138 to the medial and lateral papillary muscles 132. During atrial systole, blood flows from the left atrium to the left ventricle down the pressure gradient. When the left ventricle contracts in ventricular systole, the increased blood pressure in the chamber pushes the mitral valve to close, preventing backflow of blood into the left atrium. Since the blood pressure in the left atrium is much lower than that in the left ventricle, the flaps attempt to evert to the low pressure regions. The chordae tendineae prevent the eversion by becoming tense, thus pulling the flaps and holding them in the closed position.

FIG. 2B is a schematic representation of mitral valve prolapse as discussed above. Posterior leaflet 136 has prolapsed into left atrium 122. Moreover, certain chordae tendineae have stretched and others have ruptured. Because of damaged chordae 134a, even if posterior leaflet 136 returns to its intended position, it will eventually resume the prolapsed position due to being inadequately secured. Thus, mitral valve 130 is incapable of functioning properly and blood is allowed to return to the left atrium and the lungs.

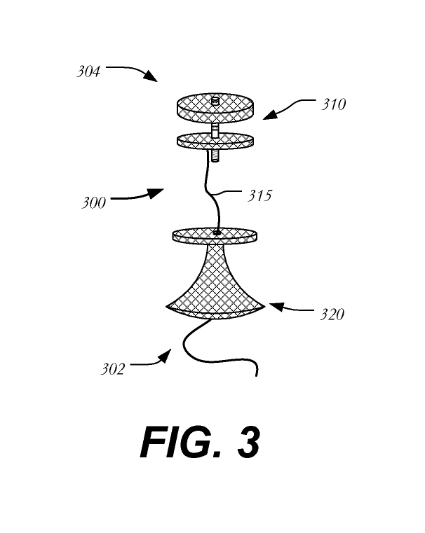

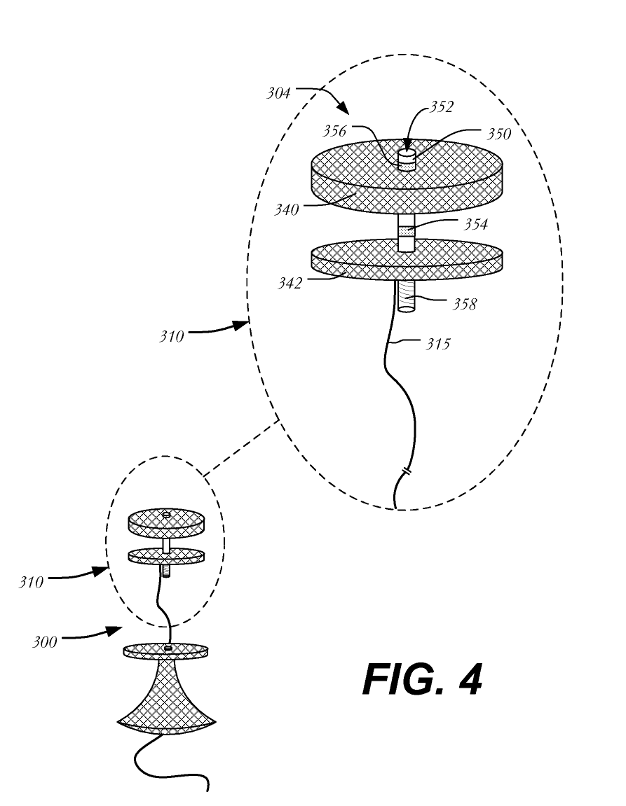

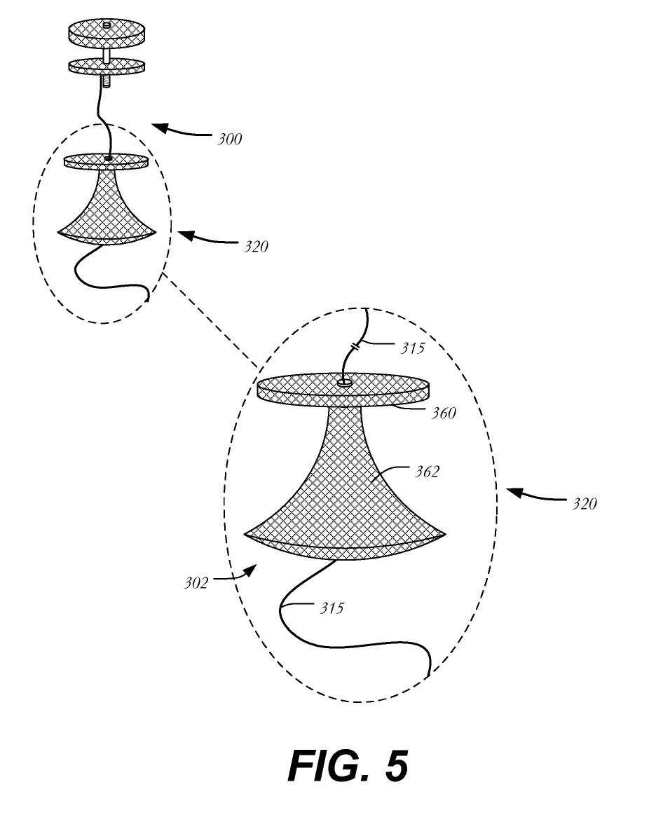

FIG. 3 is a schematic representation of chordae replacement device 300, which is capable of restoring proper function to a malfunctioning mitral valve. Chordae replacement device 300 extends between proximal end 302 and distal end 304 and generally includes first anchor 310 near distal end 304 and a second anchor 320 near proximal end 302. A filament 315 interconnects first anchor 310 and second anchor 320. One end of filament 315 is connected to first anchor 310, and the other end of filament 315 is threaded through second anchor 320 in a manner to be described below. For the sake of clarity, first and second anchors 310, 320 will be described separately with reference to FIGS. 4 and 5.

Details of first anchor 310 of chordae replacement device 300 are shown and described with reference to FIG. 4. First anchor 310 generally includes a pair of bodies, first body 340 near distal end 304 and second body 342 proximal to first body 340. First and second bodies 340,342 each may be formed of a plurality of braided strands forming a mesh which, in the deployed condition, has a generally cylindrical shape. It will be understood that bodies 340,342 may be formed of other shapes such as cubes, plates, spheres, etc. The strands forming the braid may have a predetermined relative orientation with respect to one another (e.g., a helical braid). The ends of the strands may be affixed to one another to prevent unraveling by any suitable means such as soldering, brazing, welding, gluing, tying, or clamping. Moreover, bodies 340,342 may comprise a plurality of layers of braided fabric and/or other suitable material such that bodies 340,342 are capable of at least partially inhibiting blood flow in order to facilitate the formation of thrombus and epithelialization.

Bodies 340,342 may be formed, for example, of a braided fabric mesh of a shape-memory material, of a super-elastic material, of a bio-compatible polymer, or of another material that is capable of collapsing and expanding. In the embodiments depicted in FIGS. 3-5, bodies 340,342 comprise a braided metal that is both resilient and capable of heat treatment to substantially set a desired preset shape (e.g., the relaxed configurations shown in FIG. 3). One class of materials which meets these qualifications is shape memory alloys. One example of a shape memory alloy is Nitinol. It is also understood that bodies 340,342 may comprise various materials other than Nitinol that have elastic and/or memory properties, such as spring stainless steel, trade named alloys such as Elgiloy® and Hastelloy®, CoCrNi alloys (e.g., trade name Phynox), MP35N®, CoCrMo alloys, or a mixture of metal and polymer fibers. Bodies 340,342 may further include collagen to promote epithelialization. Depending on the individual material selected, strand diameter, number of strands, and pitch may be altered to achieve the desired properties of bodies 340,342.

Due to the shape-memory properties, bodies 340,342 may be collapsed during delivery into the patient and re-expanded after delivery to serve as anchors. While bodies 340,342 are shown in FIGS. 3-5 as being cylindrical in the expanded or relaxed condition, it will be understood that bodies 340, 342 may form spheres, cubes or other suitable shapes.

First body 340 may be connected to second body 342 by a bar 350. Bar 350 may be hollow so as to define a lumen 352 through first and second bodies 340,342 for accepting a wire or other manipulating device as described below. Bar 350 may include a pair of marker bands 354,356 which may be formed of a radiopaque or other material to aid in ascertaining the position and/or orientation of chordae replacement device 300 during fluorescence x-ray, echocardiography or other visualization techniques. One marker band 354 may be positioned in bar 350 between first body 340 and second body 342. The other marker band may be positioned in bar 350 between first body 340 and distal end 304 of device 300. At its proximal end adjacent second body 342, bar 350 may be internally or externally threaded, as of 358, for releasably coupling to certain elements of a delivery device. Alternatively, bar 350 may include a ring, hook or any other suitable mechanism for releasably coupling with a delivery device.

Filament 315 is shown attached to second body 342. In some variations, filament 315 may instead be attached to first body 340, to bar 350. Additionally, filament 315 may be passed through lumen 352 of bar 350 and coupled to any of the elements of first anchor 310. Filament 315 may include a polymer such as polytetrafluoroethylene (PTFE), commonly known by the brand name TEFLON®, or other suitable metallic or polymeric materials that are biocompatible but not biodegradable, such as those used, for example, in making sutures.

Details of second anchor 320 of chordae replacement device 300 are shown and described with reference to FIG. 5. Second anchor 320 includes a pair of bodies 360, 362 formed of any of the materials and shapes described above with reference to first and second bodies 340,342. Though bodies 360,362 are shown as formed of separate portions, it will be understood that a single body may be used having a spindle-like shape. Additionally, bodies 360,362 are shaped to accommodate the outer of wall of the heart apex. Filament 315 is threaded through disk-like third body 360, which is, in turn, attached to a conical fourth body 362. Filament 315 may pass through the centers of bodies 360,362 and extend past body 362 as shown.

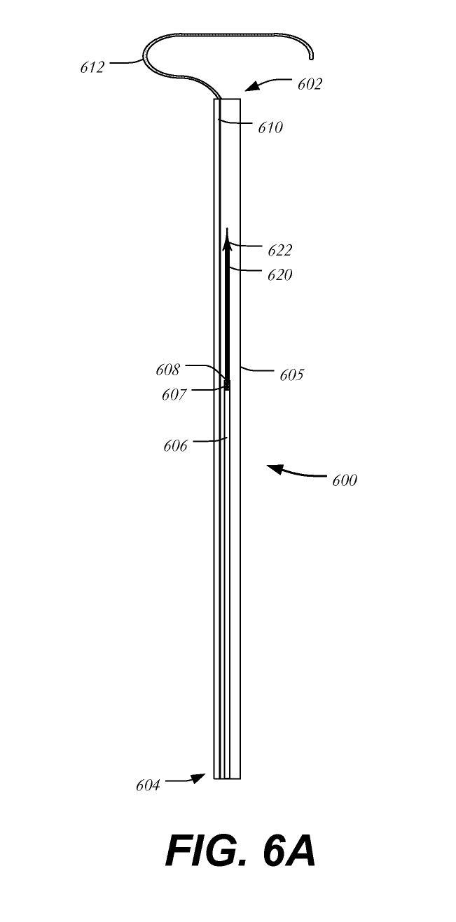

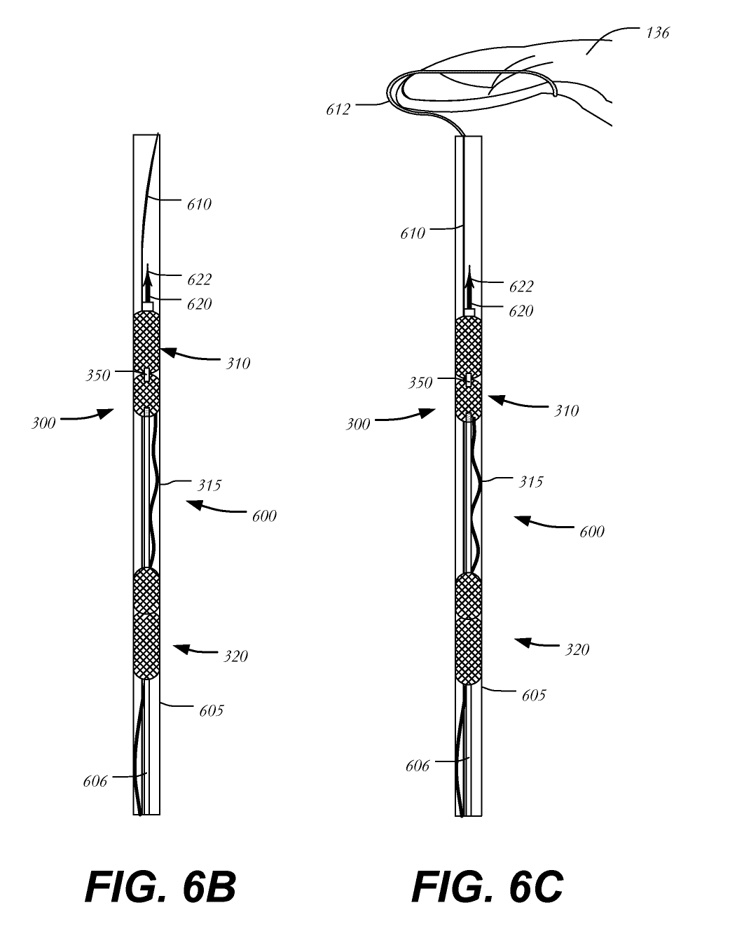

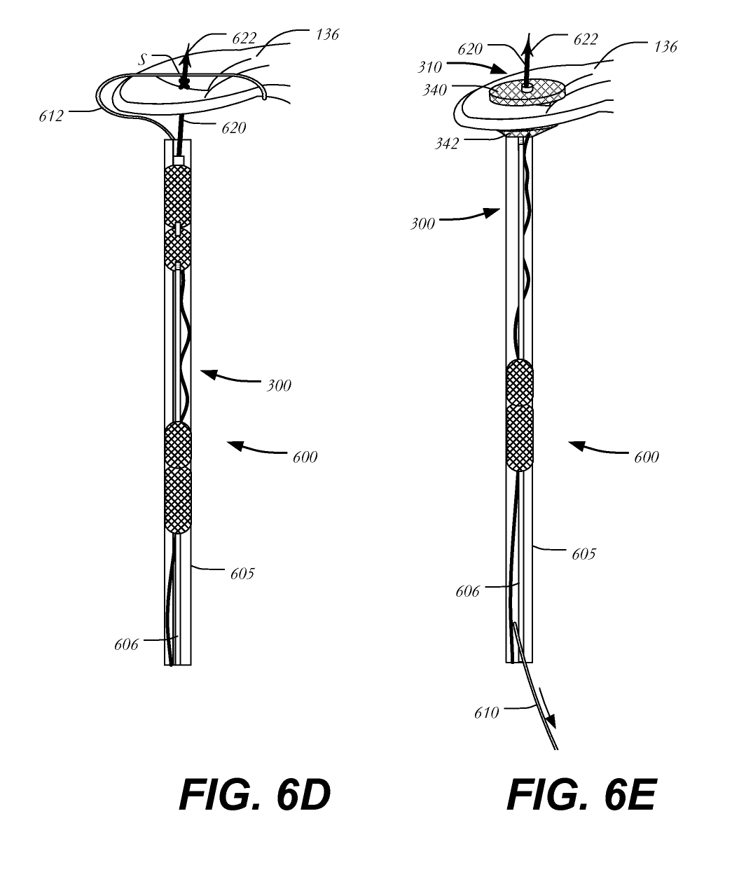

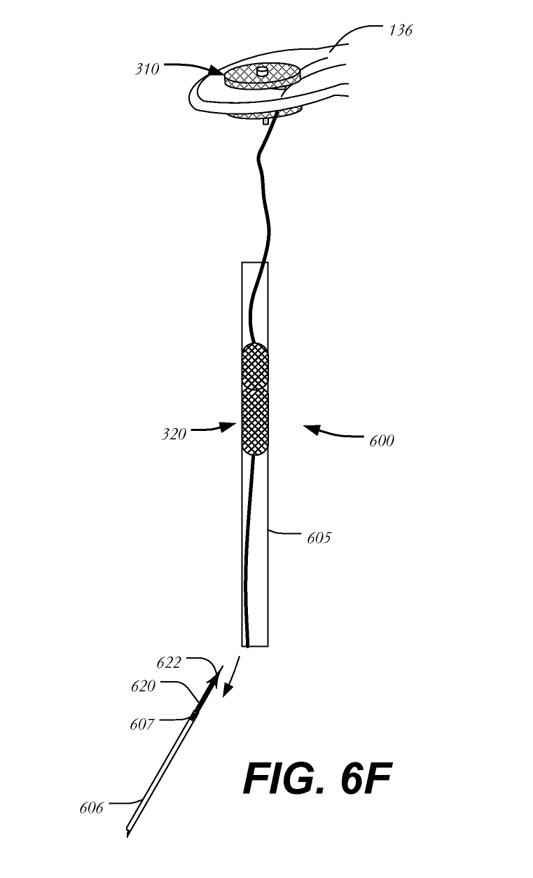

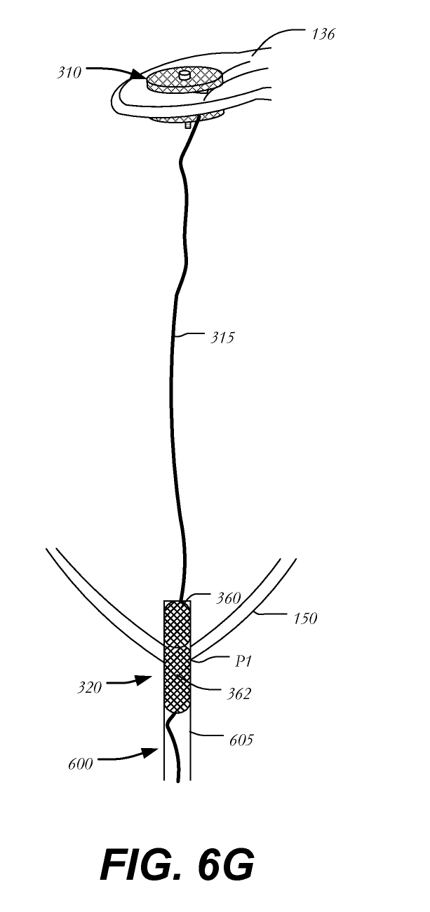

FIG. 6A illustrates a delivery system 600 for delivering chordae replacement device 300 to the vicinity of the native mitral valve and deploying first anchor 310 and second anchor 320 at their respective positions. Delivery system 600 extends between leading end 602 and trailing end 604 and includes outer shaft 605 and inner sheath 606, inner sheath 606 being disposed within outer shaft 605 and translatable relative thereto. Inner sheath 606 terminates in coupling mechanism 607, which is complementary to the coupling mechanism of chordae replacement device 300. That is, inner sheath 606 may include internally or externally threaded units at its leading end so as to threadedly engage with threaded portion 358 of chordae replacement device 300. Alternatively, inner sheath 606 may include another structure for mating with the coupling mechanism in chordae replacement device 300. Inner sheath 606 may include a lumen therethrough that is sized to accept piercing wire 620, which may be formed as a rigid metallic rod terminating in a sharp lance 622. Piercing wire 620 may be translatable relative to both inner sheath 606 and outer shaft 605. Delivery system 600 further includes retainer 610, which is translatable within outer shaft 605 and disposed adjacent inner sheath 606. As seen in FIG. 6A, retainer 610 may be formed of a shape-memory material that is configured to form a hook 612 when released from within outer shaft 605.

The use of delivery system 600 in conjunction with chordae replacement device 300 will be described with reference to FIGS. 6B-6G. FIG. 6B illustrates chordae replacement device 300 loaded within delivery system 600. In the loaded configuration, chordae replacement device 300 is disposed within outer shaft 605 with inner sheath 606 coupled to chordae replacement device 300, forming a single lumen that extends through inner sheath 606 and bar 350. As shown, second anchor 320, filament 315 and retainer 610 are all disposed to one side of inner sheath 606 and within outer shaft 605 (e.g., inner sheath 606 shown as extending behind second anchor 320). It will be understood, however, that the present configuration is merely exemplary and that numerous modification may be made and that certain elements may be made to telescope within others as desired.

As an initial step, an entry point may be identified and marked at position P1 near the apex of heart 100 for transapical delivery of delivery system 600 as shown in FIG. 1. An optional purse string suture may be made near the apex to identify and secure the entry point. An incision may then be made in the apex using a needle or other device to create an entry point and delivery system 600 may be inserted through the entry point into left ventricle 124. The needle may be inserted through outer shaft 605 or the two devices may be used sequentially (e.g., delivery system 600 may be introduced after removal of the needle). Delivery system 600 may then be advanced through left ventricle 124 to the prolapsed leaflet. FIG. 6C illustrates delivery system 600 being advanced to posterior leaflet 136, though it will be understood that delivery system 600 may be used to repair either or both leaflets. For the sake of clarity, the papillary muscles, the native chordae tendeneae and other structures of the heart are not shown. Retainer 610 may be pushed distally out of outer shaft 605 and allowed to return to its relaxed shape to form hook 612 as shown to support leaflet 136. Once hook 612 has been placed so as to adequately support the leaflet, the physician and/or user may begin the deployment of anchors 310 and 320.

Specifically, with leaflet 136 held and supported by hook 612, piercing wire 620 may be distally advanced through inner sheath 606 and bar 350, and lance 622 made to pierce through leaflet 136 to create incision “S” (FIG. 6D). Retainer 610 may then be proximally pulled through outer shaft 605 and removed from the heart (FIG. 6E). The leading end of delivery system 600 may be pushed against the underside of leaflet 136 and inner sheath 606 may be distally advanced to position bodies 340,342 near the leaflet while they are still constrained within outer shaft 605. Inner sheath 606 may then be distally advanced further, pushing first body 340 of anchor 310 over piercing wire 620 through incision “S” so that first body 340 is allowed to expand on the distal side of leaflet 136. Outer shaft 605 may be partially retracted while holding inner sheath 606 in place to deploy second body 342 out from outer shaft 605 and allow it to self-expand against the proximal side of leaflet 136. When bodies 340,342 expand, they effectively sandwich leaflet 136, securing anchor 310 to the leaflet.

Inner sheath 606 then may be unfastened from bar 350 (e.g., by rotating inner sheath 606 relative to second body 342), releasing first anchor 310 from the delivery system. Piercing wire 620 and inner sheath 606 may be removed from outer shaft 605 (FIG. 6F) leaving first anchor 310 secured to leaflet 136 and second anchor 320 still collapsed within outer shaft 605. At this juncture, filament 315 will extend out from outer shaft 605 to its connection to first anchor 310. Outer shaft 605 may then be proximally retracted toward entry position “P1” in heart wall 150 until third body 360 is positioned near the interior of heart wall 150 and fourth body 362 is positioned near the exterior of heart wall 150 (FIG. 6G). As outer shaft 605 is retracted, filament 315 will deploy therefrom so that a continuous filament extends from first anchor 310 into outer shaft 605 and proximal of second anchor 320. For the sake of clarity, only a portion of heart wall 150 and leaflet 136 are shown in FIG. 6G. Second anchor 320 may then be urged forward out of outer shaft 605 so that third body 360 expands on the interior of heart wall 150 and outer shaft 605 may be slightly retracted until fourth body 362 is deployed and expands on the exterior of heart wall 150. It will be understood that second anchor 320 may be urged forward out of outer shaft 605 using any suitable rod or wire. Alternatively, fourth body 362 may include a coupling mechanism or other mating feature such as described with reference to second body 342 and may couple to a rod that is disposed within outer shaft 605. Inner sheath 606 may also be configured to deploy second anchor 320. Regardless of the deployment method, bodies 360 and 362 may be configured to epithelialize and seal the entry point.

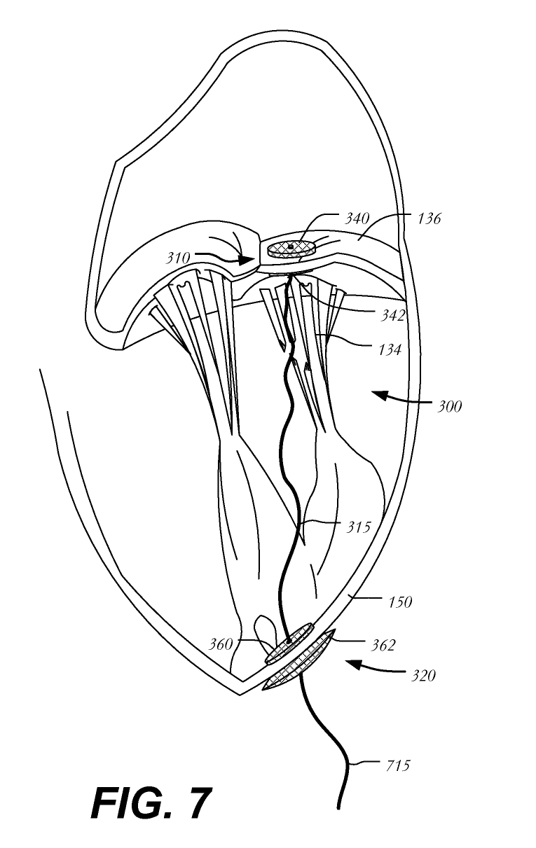

FIG. 7 illustrates chordae replacement device 300 showing first anchor 310 secured to leaflet 136 and second anchor 320 secured to heart wall 150 near the apex of heart 100. Delivery system 600 has now been completely removed from the heart. Filament 315 extends from first anchor 310 to and through second anchor 320 to the exterior of the heart. Filament 315 may also extend to the exterior of the patient for grasping by the surgeon. As seen in FIG. 7, filament 315 is not yet tensioned and includes slack between first and second anchors 310,320.

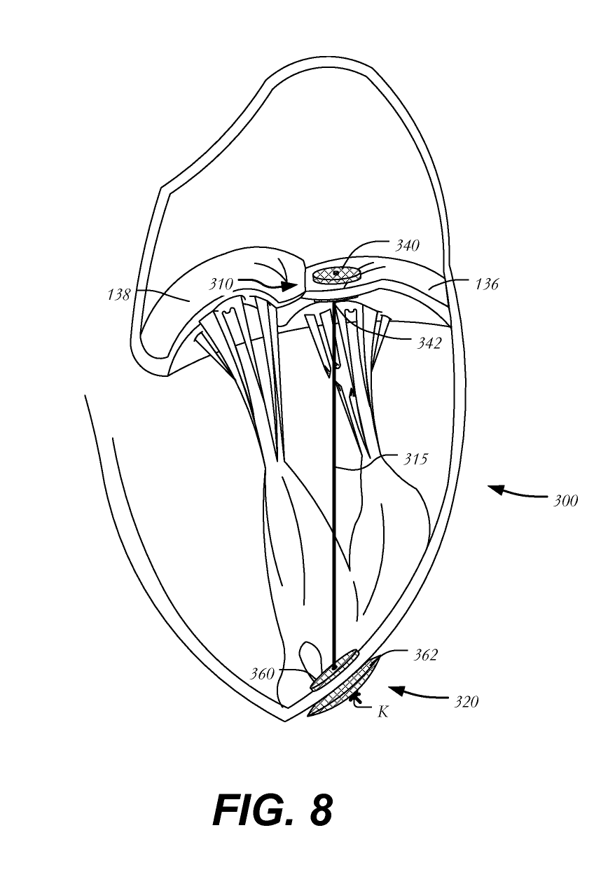

As a final step, filament 315 may be tensioned by pulling on its free tail end 715 until filament 315 is sufficiently taut while leaflet 136 is in its proper closed position (e.g., when leaflet 136 properly coapts with leaflet 138). Once leaflet 136 is in the proper position, a surgeon may form knot “K” of filament 315 adjacent fourth body 362 to maintain the tension in the filament and prevent it from slipping through anchor 320 (FIG. 8). The knotting of filament 315 may be carried out in conjunction with fluorescence x-ray, echocardiography or other visualization techniques to assure that leaflet 136 does not travel beyond in its proper closed position.

In use, posterior leaflet 136 may now be capable of deflecting downward toward the left ventricle to open mitral valve 130 during atrial systole and allow blood to flow from the left atrium to the left ventricle. During ventricular systole, filament 315 acts as a chordae tendeneae 134 to prevent leaflet 136 from prolapsing into the left atrium. As a result, leaflets 136,138 properly close to prevent regurgitation of blood back into the left atrium, allowing the mitral valve to properly function as a one-way valve.

It will be appreciated that the various dependent claims and the features set forth therein can be combined in different ways than presented in the initial claims. For example, a chordae replacement device may include more or fewer bodies than described. Furthermore, one or more bodies may be replaced by hooks or other securing elements. Additionally, it will be understood that multiple sheaths or delivery systems may be employed to deliver the chordae replacement device. For example, a first sheath may be used to insert the first anchor, which may then be retracted and replaced with a second sheath to deploy the second anchor. In another example, the hook and anchors may be introduced in separate sheaths. It will also be appreciated that any of the features described in connection with individual embodiments may be shared with others of the described embodiments.

In some embodiments, a chordae replacement device includes a first anchor for coupling to a native valve leaflet, a second anchor for coupling to heart tissue and a filament adapted for connection between the first anchor and the second anchor so as to limit the movement of the native valve leaflet away from the second anchor.

In some examples, the first anchor includes a first body and a second body configured to sandwich the native valve leaflet, and/or the first body and second body are substantially disk-shaped, and/or the second anchor includes a third body and a fourth body configured to sandwich the heart tissue, and/or the third body is substantially disk-shaped and the fourth-body is substantially conical, and/or the first and second anchors include a shape-memory material that is self-expandable from a collapsed condition during delivery to a relaxed condition during use, and/or the first and second anchors include braided strands, and/or the shape-memory material is nitinol, and/or the native valve leaflet is a mitral valve leaflet, and/or the heart tissue is an apex of a heart wall, and/or the filament includes Teflon, and/or the device includes a hollow bar connecting the first body to the second body.

In some embodiments, a delivery device for implanting a chordae replacement device having a first anchor, a second anchor and a filament at a deployment site in a patient includes an outer shaft, an inner sheath disposed within the outer shaft and translatable relative to the outer shaft, the inner sheath being coupleable to the first anchor and a piercing wire disposed within the inner sheath and translatable relative to the inner sheath.

In some examples, the device includes a retainer capable of supporting a native valve leaflet, and/or the retainer includes a shape-memory material capable of forming a hook at an end of the retainer when deployed from the outer shaft, and/or the inner sheath includes a coupling mechanism adapted to mate with a complementary coupling mechanism on the first anchor.

In some embodiments, a method is described of deploying a chordae replacement device at a target site, the chordae replacement device including a first anchor for coupling to a native valve leaflet, a second anchor for coupling to heart wall tissue and a filament having one end connected to the first anchor, and a free end. A delivery device is introduced to the target site, the delivery device including an outer shaft, an inner sheath disposed within the outer shaft and translatable relative to the outer shaft, and a piercing wire disposed within the inner sheath and translatable relative to the inner sheath. The native valve leaflet may be penetrated with the piercing wire and the first anchor advanced over the piercing wire to deploy the first anchor in engagement with the native valve leaflet. The delivery system may be withdrawn toward the heart wall and the second anchor deployed in engagement with the heart wall. A filament may be connected to the second anchor to define a fixed length of filament between the first anchor and the second anchor.

In some examples, the method further includes supporting the native valve leaflet with a retainer prior to the penetrating step, and/or further includes tensioning the filament by pulling the free end of the filament so that the filament is taut prior to the connecting step, and/or the connecting step includes tying a knot in the filament outside the heart wall.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A chordae replacement device, comprising:
a first anchor for coupling to a native valve leaflet;
a second anchor for coupling to heart tissue; and
a filament adapted for connection between the first anchor and the second anchor so as to limit the movement of the native valve leaflet away from the second anchor.

2. The chordae replacement device of claim 1, wherein the first anchor comprises a first body and a second body configured to sandwich the native valve leaflet.

3. The chordae replacement device of claim 2, wherein the first body and second body are substantially disk-shaped.

4. The chordae replacement device of claim 1, wherein the second anchor comprises a third body and a fourth body configured to sandwich the heart tissue.

5. The chordae replacement device of claim 4, wherein the third body is substantially disk-shaped and the fourth-body is substantially conical.

6. The chordae replacement device of claim 1, wherein the first and second anchors comprise a shape-memory material that is self-expandable from a collapsed condition during delivery to a relaxed condition during use.

7. The chordae replacement device of claim 6, wherein the first and second anchors comprise braided strands.

8. The chordae replacement device of claim 6, wherein the shape-memory material is nitinol.

9. The chordae replacement device of claim 1, wherein the native valve leaflet is a mitral valve leaflet.

10. The chordae replacement device of claim 1, wherein the heart tissue is an apex of a heart wall.

11. The chordae replacement device of claim 1, wherein the filament comprises Teflon.

12. The chordae replacement device of claim 2, further comprising a hollow bar connecting the first body to the second body.

13. A delivery device for implanting a chordae replacement device having a first anchor, a second anchor and a filament at a deployment site in a patient, the delivery device comprising:
an outer shaft;
an inner sheath disposed within the outer shaft and translatable relative to the outer shaft, the inner sheath being coupleable to the first anchor; and
a piercing wire disposed within the inner sheath and translatable relative to the inner sheath.

14. The delivery device of claim 13, further comprising a retainer capable of supporting a native valve leaflet.

15. The delivery device of claim 14, wherein the retainer comprises a shape-memory material capable of forming a hook at an end of the retainer when deployed from the outer shaft.

16. The delivery device of claim 13, wherein the inner sheath includes a coupling mechanism adapted to mate with a complementary coupling mechanism on the first anchor.

17. A method of deploying a chordae replacement device at a target site, the chordae replacement device including a first anchor for coupling to a native valve leaflet, a second anchor for coupling to heart wall tissue and a filament having one end connected to the first anchor, and a free end, the method comprising:
introducing a delivery device to the target site, the delivery device including an outer shaft, an inner sheath disposed within the outer shaft and translatable relative to the outer shaft, and a piercing wire disposed within the inner sheath and translatable relative to the inner sheath;
penetrating the native valve leaflet with the piercing wire;
advancing the first anchor over the piercing wire to deploy the first anchor in engagement with the native valve leaflet;
withdrawing the delivery system toward the heart wall;
deploying the second anchor in engagement with the heart wall; and
connecting the filament to the second anchor to define a fixed length of filament between the first anchor and the second anchor.

18. The method of claim 17, further comprising supporting the native valve leaflet with a retainer prior to the penetrating step.

19. The method of claim 17, further comprising tensioning the filament by pulling the free end of the filament so that the filament is taut prior to the connecting step.

20. The method of claim 17, wherein the connecting step includes tying a knot in the filament outside the heart wall.
